(19) United States
(12) Patent Application Publication
(10) Pub. No.: US 2026/0102143 A1
(43) Pub. Date: Apr. 16, 2026

(54) METHOD FOR ANALYZING A MEDIUM ALLOWING THE EFFECTS OF ARTIFACTS DUE TO STATIC DEFORMATION IN THE MEDIUM TO BE REDUCED

(71) Applicant: E-SCOPICS, Aix-en-Provence (FR)

(72) Inventors: Adrien BESSON, Marseille (FR); François MAURICE, Draguignan (FR)

(21) Appl. No.: 19/116,723

(22) PCT Filed: Sep. 29, 2023
(86) PCT No.: PCT/EP2023/077099

§ 371 Date: Mar. 28, 2025

(30) Foreign Application Priority Data
Sep. 30, 2022 (FR) FR2210011

(51) Int. Cl. A61B 8/00; G06T 5/20; G06T 7/246

(52) U.S. Cl. CPC A61B 8/485; A61B 8/5223; A61B 8/5276; G06T 5/20; G06T 7/248; G06T 2207/10088; G06T 2207/10101; G06T 2207/10132; G06T 2207/30056

(57) ABSTRACT

The present invention relates to a method for analysing a region of interest of a viscoelastic medium, the method including a step (200) of acquiring signals representative of the movement of at least one shear-propagation component and of a static-deformation component, and a processing step (300) for determining at least one property of the medium, the processing step comprising the following sub-steps: # determining images of the region of interest: # estimating images of movement by comparing images of the region of interest: # filtering the images of movement to attenuate the statis-deformation component; # determining a property of the medium from the filtered images of movement.

### FIELD OF THE INVENTION

The present invention relates to the general technical field of imaging a target object, or a diffuse medium such as human or animal biological tissue.

More specifically, the present invention relates to a device and a method for measuring viscoelastic properties of a biological tissue of interest.

It applies in particular, but not exclusively, to the measurement of viscoelasticity parameters of the liver of a human or an animal, this measurement being correlated with the amount of fibrosis present in the liver.

### BACKGROUND OF THE INVENTION

### 1. Generalities

In order to measure tissue viscoelastic properties, it is known to use shear wave elastography.

This technique consists of observing the movement of a shear wave through a region of interest (ROI) of a medium to determine parameters of the shear wave in the ROI, such as the propagation velocity of the shear wave, these parameters being directly related to the properties of the medium analyzed (for example the shear modulus of the medium analyzed).

For this purpose, shear waves are generated in an ROI, for example by implementing mechanical stress or acoustic stress.

The generated shear waves cause time-varying movements while moving from a generation point to multiple locations in the analyzed medium.

Different imaging techniques such as magnetic resonance imaging (or “magnetic resonance elastography”), optical imaging (or “optical consistency elastography”) or ultrasound imaging (or “ultrasound elastography”) can detect these movements.

In the particular case of ultrasound imaging, an imaging probe is used. Such an imaging probe comprises a casing and a transducer array, mechanically secured to the casing, for the emission of high-frequency ultrasound waves and the reception of acoustic echoes allowing to track the movement of the shear waves.

In particular, tracking shear wave-induced movements as a function of time at multiple locations allows an estimation of the shear velocity, which in turn allows the estimation of one (or more) properties of the analyzed medium.

Characterizing viscoelastic properties of the medium such as shear stiffness using shear velocity estimation has important medical applications because these properties are closely related to the state of the medium with respect to a pathology. Typically, at least a portion of a tissue may become stiffer than the surrounding tissues indicating the onset or presence of a disease or condition such as cancer, tumor, or fibrosis. However, conventional shear velocity imaging techniques suffer from artifacts due to the presence of static deformations-generated by probe movement and/or by movement of a patient's organ etc.—which degrade the quality of the signals acquired by the probe, making it difficult to accurately measure the viscoelastic properties of the medium. A purpose of the present invention is to propose a method for analyzing a region of interest (ROI) of a viscoelastic medium allowing the effects associated with artifacts (due to the presence of static deformations) in the determination of one (or more) property(ies) of the viscoelastic medium to be reduced.

Before presenting the invention, a description of the fundamental principles associated with shear wave elastography will be detailed in order to better understand the concepts relating to the static deformation problem mentioned above.

### 2. Theory of Shear Wave Elastography

### 2.1. Wave Propagation in Elastic Media

Fundamental principles of wave propagation in elastic media will now be recalled.

For this purpose, consider a homogeneous and isotropic elastic solid medium. The wave equation in the absence of force is written as follows:

$$ρ \frac{{\partial }^{2} \vec{u}}{\partial {t}^{2}} = ( λ + μ ) \vec{∇} ( \vec{∇} \cdot \vec{u} ) + μΔ \vec{u} ,$$

where:
   the vector \vec{u} denotes the movement at any point,
   the symbol \vec{∇} denotes the vector differential operator,
   the symbol Δ denotes the Laplacian operator,
   ρ is the density of the medium, and
   λ and μ are the first Lamé coefficient and the shear modulus of the solid, respectively.

The wave equation imposes the propagation of two types of propagation components in the medium:
the compression-propagation component (P waves) and
the shear-propagation component (S waves).

Theses propagation components, in a monochromatic approximation (plane wave) have distinct behaviors.

The compression-propagation component has a zero divergence, longitudinally polarized and propagates at a velocity cₚ such that:

$${c}_{p} = \sqrt{\frac{λ + 2 μ}{ρ}} .$$

The shear-propagation component has a zero rotational, transversely polarized and propagates at a velocity cₛ such that:

$${c}_{s} = \sqrt{\frac{μ}{ρ}} .$$

Thus, these two propagation components can propagate at different velocities, depending on the relative values of the two Lamé coefficients. In a biological tissue, which is almost incompressible (λ>>μ), the velocities cₚ and cₛ of the compression and shear propagation components have very different values, typically around 1500 m/s for the compressional wave and around 1 to 5 m/s for the shear wave.

In addition, since biological tissues are almost incompressible, the compression-propagation component is of negligible amplitude compared to the shear-propagation component and it is possible to consider that the movements observed in the context of elastography techniques are only related to shear effects.

It is therefore understood that the compression-propagation component is that used by conventional ultrasound imaging (allowing to achieve an acceptable spatial resolution with frequencies of the order of MHZ) used to image the movements within the framework of the invention, and the shear-propagation component is that on which the estimation of the tissue viscoelastic parameters is based in elastography methods.

### 2.2. Harmonic, Transient and Passive Elastography

With reference to FIG. 1, the principle of harmonic elastography is based on the use of a mechanical source to generate—in a medium—a mechanical wave including a shear-propagation component CIS, CIS' (and a compression-propagation component CMP, CMP′ whose amplitude is negligible in biological tissues) at low frequency, the propagation of which is monitored by ultrafast ultrasound imaging.

In particular, FIG. 1 illustrates the generation of a shear-propagation component CIS and a compression-propagation component CMP during compression of the medium by a probe S.

Monitoring the propagation of the shear-propagation component over time allows to find its propagation velocity and the Young's modulus of the medium.

The underlying idea is based on a simple rheological model. Indeed, if it is assumed that the medium is almost incompressible (an approximation valid in the case of biological tissues), then the shear modulus (and therefore the shear wave velocity) is proportional to the Young's modulus of the medium as follows:

$$μ = \frac{E}{3} ⇒ E = 3 ρ {c}_{s}^{2} .$$

Thus, measuring the velocity of the shear-propagation component allows to estimate the Young's modulus of the medium.

Ultrasound tissue Doppler, combined with ultrafast ultrasound to avoid any aliasing problems, allows to monitor the shear-propagation component and thus measure its velocity to find Young's modulus.

Different techniques for generating the shear-propagation component have been explored in the prior art.

A first technique uses a harmonic source external to the transducer array to generate the shear-propagation component. The main problem with this technique lies in the use of an external vibration source which significantly increases the complexity of implementing the measurement.

A second technique—called transient elastography technique—popularized by the “Vibration Controlled Transient Elastography” (VCTE™) method implemented in the system Fibroscan® (Echosens, Paris, France), solves this problem by generating the shear-propagation component using the vibration of a piezoelectric element, which is then used to track the propagation of the shear-propagation component. The strength of the device is that, by symmetry effects, the longitudinally polarized shear-propagation component, that is to say the near-field term, is isolated from the others and captured by Doppler processing along the line recorded by the piezoelectric element. Thus, the velocity of the shear-propagation component is measured by simple Doppler processing operated along the line recorded by the piezoelectric element. However, this second elastography technique developed in Fibroscan does not allow two-dimensional elastography and is not suitable for capturing changes in hardness within the medium.

A third technique, called ARFI or Supersonic Shear wave, depending on the implementation, consists of generating a shear wave inside the tissue itself using a high-intensity focused ultrasound beam that generates a mechanical force called ‘radiation’ mechanical force by non-linear effect. The same ultrasound transducer is used to generate the shear wave and to measure its movement subsequently by ultrafast acquisition. This technique has the advantage of not generating static deformation of the 20 medium by itself, but does not prevent the static deformations of passive elastography (for example those resulting from heartbeats) described below from interfering with the generated wave.

A fourth technique—called passive natural wave elastography-involves exploiting shear waves naturally induced by the body, for example by the heart on the liver during a cardiac cycle. The original technique is based on a diffuse movement field hypothesis: the shear-propagation components propagate in all directions. Under this hypothesis, the time derivative of the spatial cross-correlation of the movement field at a point is related to causal and anti-causal Green's functions at that point. The spatio-temporal properties of these Green's functions can be used to estimate the Young's modulus of the medium. In 30 this context, the presence of static deformations induces a bias in the cross-correlation measurement and therefore in the hardness measurement. No solution for reducing the effects associated with static deformations has been developed for this third technique, although such a solution is necessary for passive liver elastography applications where the heart induces strong static deformations.

### 2.3. Problem of Static Deformations

Tissue Doppler allows the movement to be measured at each point in the medium, as a function of time. In the case of passive and transient elastography, this movement is a sum of three components:
the two compression and shear propagation components described in 2.1;
a static-deformation component described below.

The static-deformation component is induced by a stress on the medium caused:
either by the probe,
either by an external shear wave generator such as an external vibrator,
or by an organ.

In the context of the present invention, the term “static-deformation component” or “quasi-static-deformation component” means a deformation of the medium induced by the application of a stress—for example from the surface of the medium—and revealing non-propagative shear phenomena. This static-deformation component being different at each instant, it is added to the propagation components in the measured movement. It is therefore problematic for all methods based on the measurement of propagation components, for example passive or transient elastography techniques.

In biological tissues, this component is strong near the source of stress, and zero infinitely far from the source of stress.

Such a static-deformation component may be due to the movements of the probe, and/or to the movements of an organ-such as the heart with each heartbeat or the lungs-etc. In the following, the static deformation problem will be presented with reference to a probe including:
a casing,
an array of transducers housed in the casing and configured to
   emit excitation ultrasound waves towards the medium to be analyzed (organ, biological tissue, etc.), and
   receive acoustic echoes (that is to say ultrasound waves reflected by the different interfaces of the medium to be analyzed), and
an exciter housed in the casing and configured to:
   generate vibrations, and
   transmit them to the probe
in order to mechanically produce a shear wave, the observation of which allows the measurement of properties of the medium to be analyzed.

FIG. 2a illustrates a probe S at rest placed on the medium M to be analyzed.

When the exciter is activated to generate and transmit vibrations to the probe S (to generate the shear-propagation component), these vibrations cause successive support (FIG. 2b) and release (FIG. 2c) stresses of the probe S on the medium, generating local movements and static deformations.

As shown in FIGS. 2b and 2c, in the reference frame of the probe S, the movement of a point (such as point A) located on the surface of the medium relative to the probe is zero (by continuity), while the amplitude of the movement of a point (such as point B, or point C) located under the surface of the medium relative to the probe increases with depth, and tends towards an asymptote corresponding to the amplitude of the movement of the probe for the deepest points (such as point D).

In order to better understand this phenomenon, it is possible to use a physical model of the medium as illustrated in FIG. 3 and which consists of a stack of springs whose stiffness coefficients CR1, CR2, CR3 increase as a function of depth, due to the boundary conditions of the problem.

In an incompressible medium, the stiffness coefficient of these springs is related to the shear stresses and depends on the Young's modulus of the medium, as well as on the geometry of the problem.

From the physical model illustrated in FIG. 3, a study of the movement of points B, C, located at different depths in the medium can be carried out, when a force F applied by the probe induces a movement ΔL from a point A on the surface of the medium.

By a force balance, it is possible to deduce that the compressions ΔL₂ and ΔL₃ at points b and c are expressed by the following formulas:

$$Δ {L}_{2} = ( 1 - \frac{{CR}_{2} {CR}_{3}}{{CR}_{1} {CR}_{2} + {CR}_{2} {CR}_{3} + {CR}_{1} {CR}_{3}} ) Δ {L}_{1}$$

$$Δ {L}_{3} = \frac{{CR}_{2} {CR}_{1}}{{CR}_{1} {CR}_{2} + {CR}_{2} {CR}_{3} + {CR}_{1} {CR}_{3}} Δ {L}_{1}$$

We therefore have ΔL₃<ΔL₂<ΔL₁, with

$$Δ {L}_{2} < \frac{2}{3} Δ {L}_{1} and Δ {L}_{3} < \frac{1}{3} Δ {L}_{1} .$$

Thus, the movement-due to the application of the force close to point A-measured at each point A, B, C of the medium varies according to the depth.

For illustration purposes, FIG. 4a shows a profile of the static-deformation component (that is to say movement of the medium due to the movements of the probe and relative to the probe) as a function of depth. Such a profile generally has a logarithmic shape tending towards an asymptote.

As illustrated in FIG. 4b, this static-deformation component DS (that is to say movement of the medium due to the movements of the probe) is superimposed on the propagation components CP (that is to say movement of the medium due in particular to the shear-propagation component of FIG. 4c, the compression-propagation component being negligible), which makes it difficult to observe the shear-propagation component alone, and therefore to measure the properties of the medium to be analyzed.

Indeed, the artifact in FIG. 4a (due to the static-deformation component generated by the movements of the probe and/or by the movements of an organ of the patient etc.) is particularly troublesome because:
the temporal frequency of the movements induced by this static-deformation component DS is similar to that of the movements induced by the shear-propagation component, which makes its suppression by purely temporal filtering difficult;
the amplitude of the movements induced by this static-deformation component DS:
   is significantly higher than that of the movements induced by the underlying shear-propagation component (one to two orders of magnitude), and
   increases with depth, which is problematic because the amplitude of the movements induced by the shear-propagation component decreases with depth (through dispersion and absorption effects).

### 3. Presentation of the Prior Art

Different methods have been used to avoid this artifact.
a. A first method, implemented in the Fibroscan® system, consists of using broadband excitation, or equivalently, retaining the vibration of an external source for the generation of the shear wave.
   More specifically, the Fibroscan® system proposes to attenuate the oscillation of the external source in order to generate the shear wave over a short instant, as described in document WO 2018/078002. This attenuation (or “damping”) facilitates the separation of movements of the medium due to the static-deformation component (which is brought back to zero after an oscillation cycle), from movements of the medium due to the propagation of the shear-propagation component.
   Thus, the shear-propagation component and the static-deformation component are temporally decoupled. Shear is observed when the static-deformation component is very small in the medium.
   The major problem of this method is its low energy efficiency since much energy is wasted in generating a broadband vibration (that is to say to excite and damp the external source of generation of the shear-propagation component).
b. A second method, described in a paper-titled “Probe Oscillation Shear Elastography (PROSE): A High Frame-Rate Method for Two-Dimensional Ultrasound Shear wave Elastography”, named after DC Mellema et al.—consists of using stroboscopy in order to systematically sample the same embodiment of the static-deformation component. Thus, the static-deformation component no longer biases the measurements of the shear-propagation component.
   The problem with this method is that it imposes a temporal sampling frequency (less than twice the vibration frequency of the external source) that is too low to effectively analyze the propagation of the shear-propagation component at low frequencies, for example at 50 Hz.
   In addition, this method has proven to be rather ineffective in vivo due to its inability to remove the entire static-deformation component, as described in the paper “Probe Oscillation Shear wave Elastography: Initial In Vivo Results in Liver” by D. C. Mellema et al.
c. Finally, many methods based on filtering techniques have been explored in order to reduce parasitic movements (due to compression-propagation components but especially to those of shear in unwanted directions) from that due to the shear-propagation component of interest, in particular in magnetic resonance elastography. These methods involve in particular one-dimensional or two-dimensional filtering. An example of such methods is described in the document entitled “Spatio-temporal directional filtering for improved inversion of MR elastography images” by Manduca et al.
   A disadvantage of these methods is that they require the use of two- or three-dimensional Fourier transforms, which can be computationally expensive and pose performance problems at the film boundaries.
   Moreover, some of these methods are based on non-linear directional filtering (thresholding in the frequency domain) and can generate significant artifacts, for example phantom wave propagation in a case where there is no propagation.
d. Inspired by these methods, filtering techniques using a band-pass finite impulse response (FIR) filter have been tested without success due to their lack of robustness in separating the static-deformation component and the shear-propagation component. These unsuccessful attempts are described in the paper entitled “Probe Oscillation Shear wave Elastography: Initial In Vivo Results in Liver” by D. C. Mellema et al. which teaches the person skilled in the art that the use of a finite impulse response filter is not suitable for filtering the static-deformation component. Filtering techniques using an infinite impulse response (IIR) filter have also been considered. However, infinite impulse response (IIR) filters generate phase distortions and have transient effects, generating significant artifacts in the propagation velocity measurement of the shear-propagation component.

A purpose of the present invention is to propose a method for analyzing a region of interest of a medium allowing to overcome at least one of the aforementioned disadvantages.

### BRIEF DESCRIPTION OF THE INVENTION

To this end, the invention proposes a method for analyzing a region of interest of a viscoelastic medium, the method including a phase of acquiring signals representative of the movement of at least one shear-propagation component and of a static-deformation component, the acquisition phase comprising the following steps:
emitting acquisition signals in the viscoelastic medium successively over time,
detecting and recording reception signals received from the viscoelastic medium, each reception signal being associated with a respective acquisition signal,
   remarkable in that the method further comprises a processing phase for determining at least one property of the medium, said processing phase comprising the following sub-steps:
determining images of the region of interest of the medium from the reception signals, each image being determined from a respective reception signal and comprising information representative of the region of interest at different times,
estimating images of movement by comparing images of the region of interest, each movement image including information representative of the shear-propagation component and a static-deformation component,
filtering the images of movement to attenuate the static-deformation component,
determining said and at least one property of the medium from the filtered images of movement.

In the context of the present invention, the term “mechanical wave” means a wave comprising:
shear-propagation component generated either naturally (that is to say passive elastography in which the shear-propagation component is generated by an organ such as the heart during a cardiac cycle), or artificially (that is to say harmonic or transient elastography in which the shear-propagation component is generated by the force of ultrasonic radiation, or by an external source-such as an inertial vibration exciter of the type described in WO 2022/084502,
and a compression-propagation component, also generated either naturally or artificially, and whose amplitude in biological tissues is low relative to the shear-propagation component. The amplitude of this compression-propagation component being very low relative to the amplitude of the shear-propagation component, it will be neglected in the remainder of the description.

In the context of the present invention, “shear wave” or “shear-propagation component”, means a “low frequency” wave polarized transversely in the far field, that is to say a wave producing movements of the medium in a direction perpendicular to the direction of propagation of the wave and whose frequency is typically comprised between 10 Hz and 1500 Hz.

In the context of the present invention, the term “compressional wave” or “compression-propagation component”, means a “low frequency” wave polarized longitudinally in the far field, that is to say a wave producing movements of the medium in a direction parallel to the direction of propagation of the wave and whose frequency is typically comprised between 10 Hz and 1500 Hz.

In the context of the present invention, the term “static-deformation component” means the deformations induced by a stress on the medium. In biological media, this static-deformation component is related to shear phenomena.

In the context of the present invention, the term “ultrasound wave” means a longitudinally polarized “high frequency” compressional wave, that is to say a wave producing movements of the medium in a direction parallel to the direction of propagation of the wave and whose frequency is typically comprised between 15 kHz and 20 MHz. In the context of the invention, this ultrasound wave is used as a means for acquiring the images of movement used to monitor the propagation of the shear-propagation component.

For the purposes of the present invention, the term “group velocity” of a wave means a velocity at which an overall shape of the envelope of the amplitudes of the wave, known as the envelope of the wave, propagates in space.

Preferred but non-limiting aspects of the analysis method according to the invention are as follows:
the filtering step may comprise the application of a finite impulse response filter the characteristics of which are defined according:
   to an average frequency of the shear-propagation component comprised between 10 Hz and 1500 Hz, and
   to a maximum group velocity of the shear-propagation component in the region of interest;
the finite impulse response filter can comprise a passband and a stopband:
   the passband being defined at least by a lower bound K₋₆, said lower bound K₋₆ being equal to a ratio between the average frequency of the shear component and the maximum group velocity of the shear component, the attenuation in the passband being less than or equal to 6 dB,
   the stopband being defined at least by a stop bound K₋₃₀ proportional to the lower bound K₋₆, the attenuation in the stopband being greater than or equal to 30 dB, and the ratio between the lower bound K₋₆ and the stop bound K₋₃₀ defining a stiffness coefficient r of the finite impulse response filter, said stiffness coefficient being comprised between 1 and 4.5;
the minimum and maximum velocities can be determined based on predefined minimum and maximum hardnesses in the region of interest;
the average frequency of the shear component can be estimated from a signal representative of a time spectrum of the images of movement;
the method may further comprise an excitation phase including a step of generating the shear-propagation component by applying to the viscoelastic medium an excitation having the form of a low frequency impulse which has a central frequency f comprised between 10 Hz and 1500 Hz;
the finite impulse response filter may be of the highpass type in a useful frequency range comprised between 0 and 50 m⁻¹;
said finite impulse response filter can be obtained by combining an all-pass type filter and a lowpass type filter in said useful frequency range;
the lowpass filter can be a rectangular window lowpass filter or a filter whose impulse response is such that its maximum value is less than or equal to 2 times its average value.

### BRIEF DESCRIPTION OF THE DRAWINGS

Other advantages and characteristics of the analysis method according to the invention will emerge more clearly from the following description of several variant embodiments, given as non-limiting examples, from the appended drawings in which:

FIG. 1 is a schematic representation illustrating the generation of compression and shear waves during compression of a medium,

FIG. 2a is a schematic representation of a probe at rest positioned on a medium to be analyzed,

FIG. 2b is a schematic representation illustrating the generation of static deformations by pressing the probe on the medium to be analyzed,

FIG. 2c is a schematic representation illustrating the generation of static deformations by releasing the probe on the medium to be analyzed,

FIG. 3 is a schematic representation of a spring model,

FIG. 4a is a schematic representation of a movement profile induced by the static-deformation component as a function of a propagation direction of the shear-propagation component,

FIG. 4b is a schematic representation of a movement profile induced by the superposition of the static-deformation component and the shear-propagation component, as a function of a propagation direction of the shear-propagation component,

FIG. 4c is a schematic representation of a movement profile induced by the shear-propagation component as a function of a propagation direction of the shear-propagation component, once the static-deformation component, indicated DS in FIG. 4b, has been attenuated using the method described according to the invention,

FIG. 5 is a schematic representation of an analysis device according to the invention,

FIG. 6 is a schematic representation of a variant embodiment of the analysis method according to the invention,

FIG. 7 is a schematic representation of an impulse response of an exemplary filter that may be used in a filtering sub-step of the analysis method.

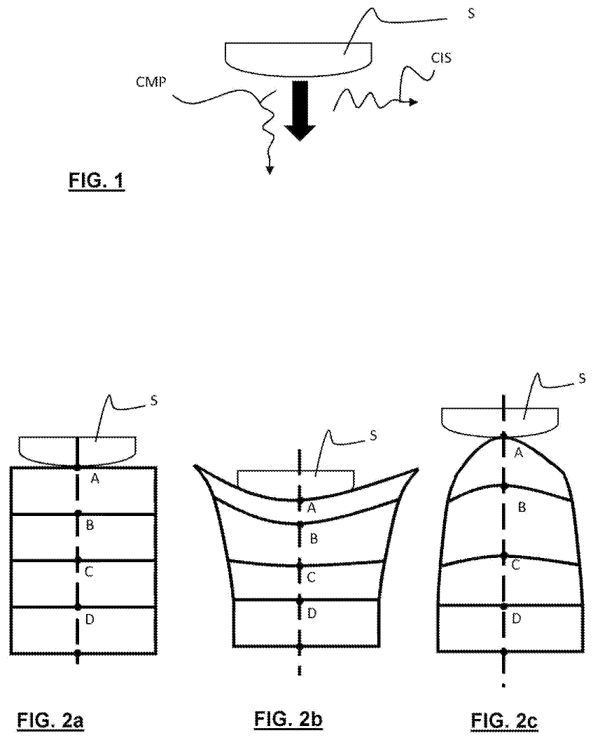

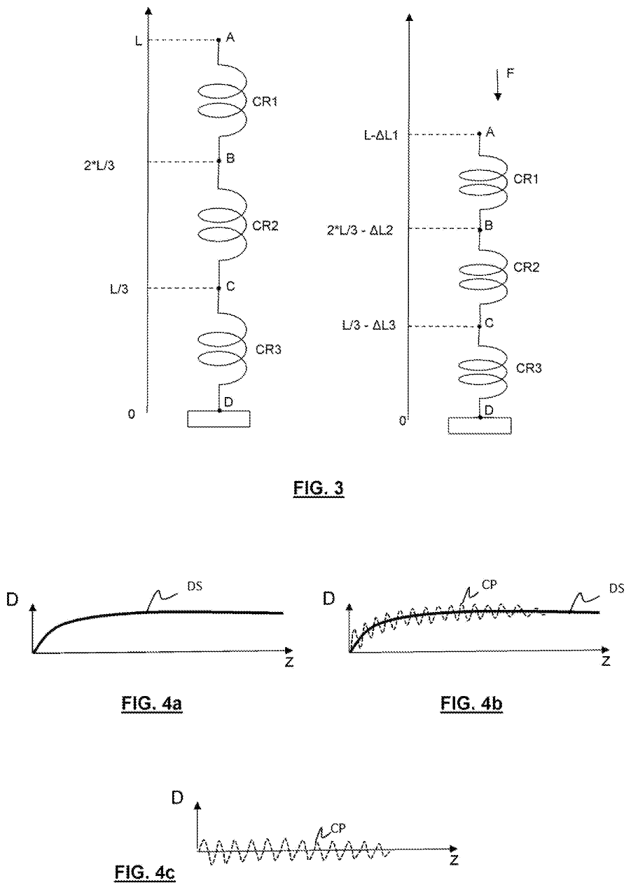

### DETAILED DESCRIPTION OF THE INVENTION

Different embodiments of the analysis method will now be described in more detail according to the invention with reference to the figures. In these different figures, equivalent elements are designated by the same numerical reference.

In the following, the analysis method will be described with reference to the processing of data acquired using an ultrasound elastography probe allowing:
to generate at least one low-frequency elastic wave—called a “shear wave”—in the medium, and
simultaneously with the generation of the low frequency wave:
   emit high-frequency ultrasound waves, and
   receive acoustic echoes due to reflections of ultrasound waves in the medium,
      in order to observe the propagation of the low-frequency elastic wave in the medium. It is obvious to the person skilled in the art that the analysis method can be implemented with passive elastography techniques using natural waves—in which shear waves are generated naturally by the body. In this case, the probe only comprises an array of transducers for the emission of ultrasound waves according to a high-frequency depth (15 kHz-20 MHz) and the reception of acoustic echoes in order to observe the propagation of one (or more) shear waves generated naturally by the body.

It is also obvious to the person skilled in the art that the analysis method according to the invention can be implemented with imaging techniques other than ultrasound imaging (or “ultrasound elastography”), in particular magnetic resonance imaging (or “magnetic resonance elastography”), or optical imaging («optical coherence elastography»).

### 1. Generalities

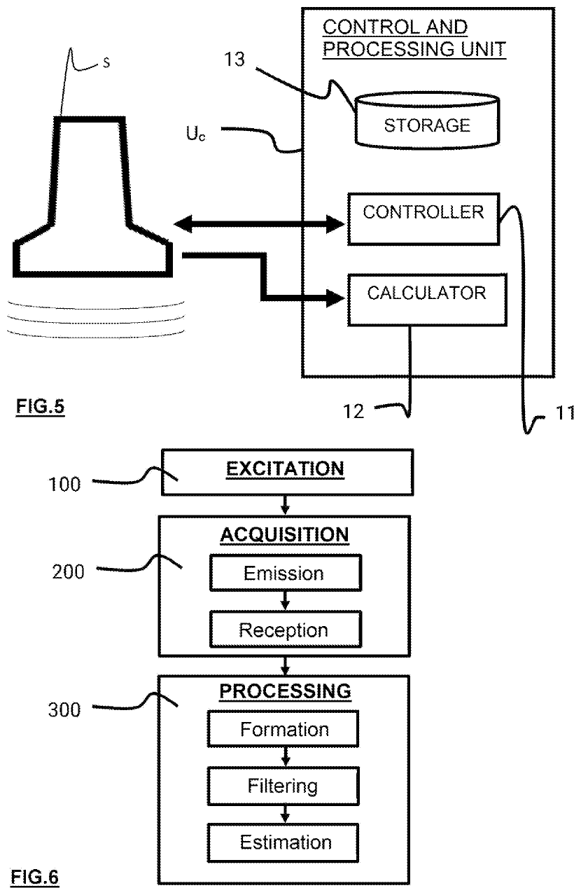

With reference to FIG. 5, an example of a device in which the method for analyzing a medium described below can be implemented is illustrated.

This device comprises:
a signal acquisition probe S, and
a control and processing unit U_{c} for:
   piloting the probe S, and
   processing signals acquired by the probe S.

### 1.1. Impulse Elastography Probe

The probe S comprises:
an array of transducers for the emission of ultrasound waves and the reception of acoustic echoes, and
an exciter for shear wave generation.

The exciter may be an inertial vibration exciter of the type described in WO 2022/084502. It is used to generate vibrations. A fixed part of the exciter is mechanically secured to the transducer array to transmit the vibrations to the probe in order to mechanically produce the shear wave necessary for measuring the viscoelastic properties of the tissue to be analyzed.

The use of an inertial vibration exciter allows to obtain a probe with limited weight and size.

The use of an inertial vibration exciter further allows the generation of a shear wave of sufficient power to measure tissue viscoelastic properties while consuming less energy than other solutions (such as solutions based on ultrasound radiation pressure). The exciter can also be external to the probe, and move it in its entirety, although this solution is not optimal from the point of view of size.

Since such a probe is known in the prior art, it will not be described in more detail below.

### 1.2. Control and Processing Unit

The control and processing unit U_{c} is connected to the probe S by wired or wireless communication means.

It allows to pilot the transducers of the probe S, and to process the data acquired by the transducers of the probe S. It also allows to activate the inertial vibration exciter for the generation of one (or more) shear wave(s) in the medium to be analyzed which can be an organ of a patient such as the liver.

More precisely, the control and processing unit U_{c} allows:
to control the inertial vibration exciter to produce a vibration enabling the generation of a shear wave in the medium to be analyzed,
to control the transducers to emit ultrasound waves to the medium to be analyzed,
to control the transducers to receive echoes reflected by the medium to be analyzed and convert them into reception signals,
to process the reception signals.

The control and processing unit U_{c} can be composed of one or more distinct physical entities, possibly remote from the probe S.

The control and processing unit U_{c} comprises for example:
one (or more) controller(s) 11, such as a Smartphone, a personal assistant (or “PDA”, acronym for “Personal Digital Assistant”), or any type of mobile terminal known to the person skilled in the art; and
one (or more) calculator(s) 12, such as one or more computer(s), one or more microcomputer(s), one or more workstation(s), and/or other devices known to the person skilled in the art including one or more processor(s), one or more microcontroller(s), one or more programmable logic controller(s), one or more application-specific integrated circuit(s), and/or other programmable circuits,
one (or more) storage unit(s) 13 including one (or more) memory(ies) which may be a ROM/RAM memory, a USB key, a memory of a central server.

In addition to storing data associated with the analysis of a medium, the storage unit 13 also allows to store programming code instructions intended to execute the steps of the analysis method described below.

### 1.3. Operating Principle

With reference to FIGS. 5 and 6, the operating principle of the imaging device is as follows.

The controller 11 causes the exciter to generate vibrations in the viscoelastic medium during an excitation step (step 100). This allows to generate a mechanical wave in the medium, including a shear-propagation component and a compression-propagation component that is negligible in the biological tissues.

The movements of the medium induced by the propagation of the mechanical wave in the viscoelastic medium are measured by ultrasound, during an observation step (step 200) concomitant with the excitation step (100).

For this purpose, the controller 11 pilots the emission of ultrasound waves (at a frequency comprised, for example, between 15 kHz and 20 MHz) by the transducers of the probe S. These ultrasound waves penetrate into the medium where they are reflected on scattering particles contained in said medium—such as, for example, collagen particles contained in the medium if it is human tissue—which allows to follow the movements of the medium. The ultrasound waves thus emitted can be “plane” (that is to say waves whose wave-front is rectilinear in an analysis plane (D, Z)) or spiral, or divergent, or else focused.

In particular, the observation step (200) comprises the following sub-steps:
the controller 11 controls the emission by the array of transducers of a succession of ultrasound wave shots into the medium (at a rate comprised between 10 and 10000 shots per second),
the controller 11 controls the detection by the array of transducers, and the recording (in real time) in the storage unit 13 of the received acoustic signals comprising the echoes generated by the ultrasound waves by interacting with the diffusing particles of the medium.

The acoustic signals recorded in the storage unit 13 are then processed (generally in delayed time) by the computer during a processing step 300.

More specifically, the processing step 300 comprises a sub-step of determining—by a conventional beamforming process—images of a region of interest of the medium contained in the observation field (that is to say the area insonified by the ultrasound waves). Each image is representative of the region of interest after a respective shot. As the shots are carried out successively over time, each image is representative of the region of interest at a different time.

These successive images of the region of interest are then compared, by correlation and in particular by cross-correlation:
either two by two
either with a reference image (which can be a previously determined movement image or an image of the region of interest obtained during a preliminary observation step), to determine images of movement of the medium.

These images of movement are representative of the propagation of the mechanical wave in the medium.

These images of movement also comprise a static-deformation component, which can significantly hamper the monitoring of the propagation of the mechanical wave, used for the determination of one (or more) properties of the medium.

This is why the inventors developed a sub-step of filtering the received acoustic signals—and in particular the images of movement—to remove the static-deformation component of the measured movements, and thus retain only the propagation component, that is to say the mechanical wave, in the images of movement.

This filtering sub-step will now be described in more detail.

### 2. Filtering Sub-Step

The filtering sub-step comprises the application of a finite impulse response filter the characteristics of which are defined according:
to an average frequency of the shear-propagation component typically comprised between 10 Hz and 1500 Hz, and
to a maximum velocity (in particular a maximum group velocity) of the shear-propagation component in the region of interest.

The average frequency of the shear-propagation component depends on the solution chosen for the generation of this shear-propagation component. However, in all cases, this average frequency is known or can be measured. For example, in the case of using an exciter, the latter is configured to generate a shear-propagation component at a desired average frequency (for example at an average frequency of 50 Hz). In this case, the average frequency of the shear-propagation component is therefore known a priori. If the solution chosen for the generation of the shear wave is natural (generation of the shear wave by an organ of the patient), then the average frequency of the shear-propagation component can be calculated from the images of movement by any technique known to the person skilled in the art.

Similarly, the maximum group velocity of the shear-propagation component in the region of interest depends on the type of region of interest being observed. However, for each type of region of interest, this maximum group velocity is known in the literature or can be measured. For example, if the region of interest consists of soft tissue (liver, spleen, prostate, breast, muscle etc.), it is known that the maximum group velocity of the shear-propagation component is of the order of 6 m/s.

Thus, the values of the average frequency and the maximum group velocity of the shear-propagation component are known and allow the dimensioning of the finite impulse response filter which will now be presented below.

### 2.1 Characteristics of the Filter

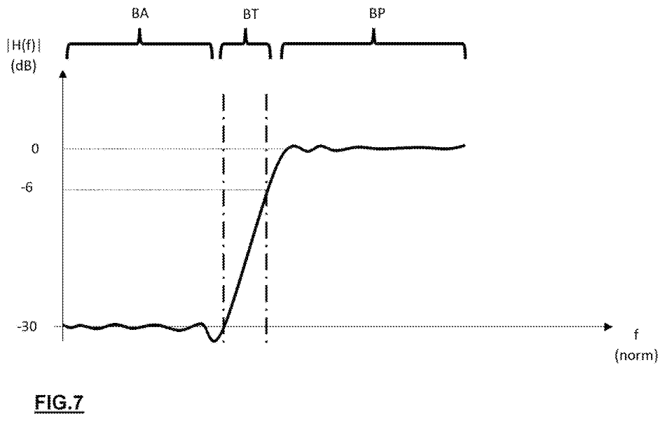

The frequency response (that is to say variation of the filter gain as a function of the frequency of the signal applied thereto as input) of an example of a filter that can be used during the filtering sub-step is illustrated with reference to FIG. 7. This frequency response is a curve representing an attenuation (in dB) as a function of a frequency (in Hz).

In this embodiment, the finite impulse response filter is a “highpass” filter.

As illustrated in FIG. 7, this frequency response (or gain plot of a Bode diagram) is composed:
of a stopband BA,
of a passband BP, and
of a transition band BT between the stopband BA and the passband BP.

### 2.1.1. Stopband

The stopband BA corresponds to a frequency range in which a signal applied to the filter input appears strongly reduced at the filter output: in this frequency range, the amplitude of the signal applied to the filter input is attenuated (to a value close to zero) at the filter output.

In the case of a highpass filter, the stopband BA can be defined by a stop bound. The stop bound—or stop wavenumber—corresponds to a ratio between a frequency and a group velocity (representative of the propagation velocity of the shear-propagation component) expressed in cycles per meter. This stop bound allows to express at what frequency the filter ceases to produce its “attenuated” effect.

In the context of the present invention, this “attenuated effect” is considered to be produced when the amplitude of a signal applied to the filter input is divided by 32 (or more) at the filter output (−30 dB). Therefore, the term K₋₃₀ will be used in the rest of the text to characterize the upper part of the stopband.

Thus, the stop bound K₋₃₀ (or stop wavenumber K₋₃₀) of the stopband BA is representative of the frequency at which the amplitude of a signal applied at the filter input is divided by 32 at the filter output (−30 dB).

In this stopband BA, the amplitude of a signal applied to the filter input is therefore attenuated by an attenuation factor FA, greater than or equal to 30 dB.

### 2.1.2. Passband

The passband BP corresponds to a frequency range in which a signal applied to the filter input appears little or not attenuated at the filter output: in this frequency range, the amplitude of the signal applied to the filter input is substantially unchanged (or slightly attenuated) at the filter output.

In the case of a highpass filter, the passband BP can be defined by a lower bound. This lower bound—or wavenumber—corresponds to a ratio between a frequency and a group velocity (representative of the propagation velocity of the shear-propagation component) expressed in cycles per meter. This lower bound allows to express at what frequency the filter ceases to produce its “passing” effect.

In the context of the present invention, this “passing effect” is considered to be produced when the amplitude of a signal applied to the filter input is divided by 2 (or less) at the filter output (−6 dB). Therefore, the term K₋₆ will be used in the rest of the text to characterize the lower part of the passband.

Thus, the lower bound K₋₆ of the passband BP is representative of the frequency at which the amplitude of a signal applied at the input of the filter is divided by 2 at the output of the filter (−6 dB).

In this passband BP, the amplitude of a signal applied to the filter input is therefore attenuated by an attenuation factor FA, less than or equal to 6 dB.

The lower bound K₋₆ corresponds to the ratio between:
the average frequency of the shear-propagation component, and
the maximum group velocity of the shear-propagation component.

As previously stated, the average frequency of the shear-propagation component is known. Similarly, the maximum group velocity of the shear-propagation component is known (of the order of 6 m/s in the case of soft tissue).

It is therefore possible to define the lower bound K₋₆ of the passband BP as a function of the type of tissue analyzed and the type of excitation considered for the generation of the shear-propagation component.

### 2.1.3. Transition Band

The transition band BT corresponds to a frequency range in which an input signal is neither strongly attenuated (stopband BA) nor substantially unchanged (passband BP). This transition band BT characterizes the filter's ability to retain or reject frequencies more or less abruptly. It is therefore representative of the filter's “reactivity”.

In this transition band, the filter is characterized by a stiffness coefficient (or “stiffness constant”) corresponding to the ratio between:
the lower bound K₋₆ of the passband BP, and
the stop bound K₋₃₀ of the stopband BA.

Thus, the stiffness coefficient is defined as the following ratio:

$$r = \frac{{K}_{- 6}}{{K}_{- 30}} .$$

This stiffness coefficient illustrates the slope of the amplitude response of the filter in the transition band between a regime where the filter attenuates strongly and a regime where the filter no longer attenuates.

The inventors determined that it was preferable for the stiffness coefficient to be less than or equal to 4.5 so that the filter would allow significant attenuation of the static-deformation component in the received acoustic signals without unacceptably impacting the low shear frequencies corresponding to high shear wave velocities, and therefore high hardnesses. The filter efficiency depends on two criteria of the transition band:
a cutoff frequency,
the stiffness coefficient.

The filter cutoff frequency corresponds to the useful operating limit frequency of the filter. More precisely, the cutoff frequency allows to express at what frequency the filter begins to produce its “passing” effect. Thus, the lower bound K₋₆ of the passband BP is representative of the filter cutoff frequency.

### 2.2. Filter Variants

Other types of filters can be used to attenuate the static distortion component contained in the signals received by the probe.

For example, a band-pass filter can be used instead of the highpass filter shown in FIG. 7.

In the case of a band-pass filter, the passband BP is further defined by an upper bound Kₘₐₓ equal to a ratio between the average frequency of the shear-propagation component and a minimum group velocity of the shear-propagation component.

The values of the average frequency and the minimum group velocity being known (measurable or known from the literature depending on the tissue to be imaged), this upper limit Kₘₐₓ is easily determined.

In all cases, the chosen filter has the behavior of a highpass type filter in a useful frequency range, for example comprised between 0 and 50 m⁻¹.

Advantageously, this finite impulse response filter can be obtained by combining an “all-pass” type filter and a “lowpass” type filter in said useful frequency range. This makes it easier to obtain an effective filter for:
the attenuation of the static-deformation component, and
the conservation of the shear-propagation component.

### 2.3. Theory of the Invention

A filtering technique along the direction of the static-deformation component is proposed, in order to drastically reduce the impact of the static-deformation component. This technique is based on the idea that the static-deformation component and the shear-propagation component have very different spatial characteristics:
short wavelengths (a few cms at most at 50 Hz) for the shear-propagation component with group velocities varying between 0.5 m/s and 6 m/s in soft tissues such as the spleen and liver.
high wavelengths (tens of μm at 50 Hz) for the static-deformation component.

### 2.3.1. Highpass FIR Filter

In one embodiment, the filtering technique is based on a highpass finite impulse response (FIR) filter (not necessarily a linear phase response filter). Its frequency characteristics are defined as a function of the average frequency of the studied shear waves f as well as the minimum and maximum group velocities of interest cₘᵢₙ and cₘₐₓ.

The average frequency of the shear wave can be estimated using the formula below: where:

$$f = \frac{{\int }_{f} {\vert V ( f ) \vert}^{2} fdf}{{\int }_{f} {\vert V ( f ) \vert}^{2} df} ,$$

V(f) is a signal representative of the time spectrum of the shear wave (for example the time Fourier transform of the images of movement).

Typical values of the average frequency of shear waves are comprised between 10 Hz and 500 Hz. The average frequency value is most often assumed to be known. For example, in the case where the shear wave is generated naturally (passive elastography), the average frequency value can be measured. In the case where the shear wave is generated artificially (transient elastography), the average frequency value is deduced from the characteristics of the mechanical source used.

The group velocity of a shear wave with frequencies in the interval [f₀−df, f₀+df] is estimated as follows:

$$c = 2 {π ( \frac{df}{dk} )}_{{f}_{0}} .$$

Typical values of shear wave group velocity are comprised between 0.5 m/s to 5.5 m/s for soft tissues such as the liver.

Interest is particularly given to two values of the wavenumber:
K₋₆ which corresponds to the value of the wavenumber (in number of cycles per meter) for which the attenuation of the filter is equal to 6 dB,
K₋₃₀ which corresponds to the value of the wavenumber (in number of cycles per meter) for which the attenuation of the filter is equal to 30 dB.

As an indication, it is recalled that the “wavenumber” (or “repetency”) is a quantity proportional to the inverse of the wavelength. The “wavenumber” is the analog, in space, of the temporal frequency and characterizes the propagation of a wave in space, as the temporal frequency does in time.

The above-mentioned attenuation is calculated based on the filter gain value in the passband as follows: where:

$${A}_{dB} ( {k}_{z} ) = 20 {log}_{10} ( \frac{H ( {k}_{z} )}{{G}_{H}} ) ,$$

H is the amplitude of the frequency response of the filter of interest and
G_{II} is a quantity representative of the filter gain in the passband.

This quantity can, for example, be the maximum of the absolute value of the frequency response in the passband or the average value of the absolute value of the frequency response in the passband.

Combined with these two values, the stiffness coefficient of the highpass filter response is defined as follows:

$$r = \frac{{K}_{- 6}}{{K}_{- 30}} .$$

This stiffness coefficient characterizes the average slope of the frequency response of the filter between these two values since we have:

$${A}_{dB} ( {K}_{- 6} ) \times r \approx {A}_{dB} ( {K}_{- 30} ) .$$

At a higher level, it can be seen as a quantifier of the effectiveness of the highpass filter.

This filter is very generic and can be produced by a person skilled in the art using finite impulse response filter synthesis tools, such as the “firwin” and “firwin2” functions of the “scipy signal processing” library of the Python programming language.

Advantageously, the stiffness coefficient of the filter is chosen to obtain sufficient filter efficiency. For example, the value of the stiffness coefficient can be chosen to be less than 4.5.

A highpass filter of this type can be made by a person skilled in the art by constructing a phase-distorting FIR filter. An example code is given below for the case of a shear wave whose group frequency is assumed to be 50 Hz.

A finite impulse response filter of 2.5 cm length is defined.

# Sampling frequency
Dz = 0.6523e−3
# Shear wave frequency
f = 50 # Hz
# Definition of the highpass filter
probe_fir_len = 2.5e−2
probe_half_fir = int(np.round(probe_fir_len/Dz − 1)/2)
probe_fir = −np.ones((2 * probe_half_fir +1,))/(2 * probe_half_fir + 1)
probe_fir [probe_half_fir] = 0
probe_fir [probe_half_fir] = 1 − 1./len(probe_fir)
# Filter frequency response
w, h_probe_fir = sig.freqz(probe_fir, fs=1/Dz, worN=8192)
# Cutoff frequency and associated stiffness coefficient
cutoff_0 = np.argmin(np.abs(20*np.log10(np.abs (h_probe_fir)/np.abs(h_probe_fir
[−1])) + 30))
cutoff_1 = np.argmin(np.abs(20*np.log10(np.abs(h_probe_fir)/np.abs (h_probe_fir
[−1])) + 6))
k_6 = w[cutoff_1]
k_30 = w[cutoff_0]
c_max = f/k_30
c_min = f/k_6
r = k_6/k_30

In this case, the characteristic quantities of the filter are as follows: K₋₆=24.6 m⁻¹, K₋₃₀=5.71 m⁻¹ and r=4.31.

The reader will appreciate that a stiffness constraint lower than 4.5 is difficult to achieve using the “firwin2” function since the “firwin2” function imposes a phase linearity constraint (or equivalently antisymmetry of the filter) to ensure that the filter does not have phase distortion.

An example below illustrates this difficulty in a case similar to above:

probe_fir_len = 2.5e−2
probe_half_fir = int(np.round(probe_fir_len/Dz − 1)/2)
numtaps = 2 * probe_half_fir
cutoff_1 = k_min
cutoff_0 = k_min/16
hp = sig.firwin2(numtaps, [0.0, cutoff_0, cutoff_1, 1.0/2/Dz], [0, 10 **(−30/20), 1.0,
1.0], fs = 1/Dz,
antisymmetric = True, window = ‘rect’)
w, h = sig.freqz (hp, fs=1/Dz, worN=2048*4)
cutoff _0_meas = np.argmin(np.abs(20*np.log10(np.abs(h)/np.abs (h[−1])) + 30))
cutoff _1_meas = np.argmin (np.abs(20*np.log10(np.abs(h)/np.abs (h[−1])) + 6))
k_6 = w[cutoff_1_meas]
k_30 = w[cutoff_0_meas]
c_max = f/k_30
c_min = f/k_6
r = k_6/k_30
slope = 1./alpha

In this case, the associated stiffness coefficient is 16.7.

To reduce the stiffness coefficient, it is possible to vary the filter length or other parameters. Thus, the example below allows a stiffness lower than 4.5 to be achieved using “firwin” and a filter of size 3.2 cm.

# Sampling frequency
Dz = 0.6523e−3
# Shear wave characteristics
f = 50 # Hz
c_min = np.sqrt (12/3) # m/s
c_max = np.sqrt (500/3) # m/s
k_max = f/c_min # m\hat{ }−1
k_min = f/c_max # m\hat{ }−1
# Definition of the highpass filter
probe_fir_len = 3.2e−2
probe_half_fir = int(np.round(probe_fir_len/Dz − 1)/2)
numtaps = 2 * probe_half_fir
hp = sig.firwin(numtaps+1, k_max, width=2*(k_max−k_min), fs = 1/Dz,
pass_zero=‘highpass’)
# Frequency response of the filter
w, h = sig.freqz(hp, fs=1/Dz, worN=2048*4)
# Wavenumbers k_6 and k_30 and associated stiffness coefficient
cutoff_0_meas = np.argmin(np.abs(20*np.log10(np.abs(h)/np.abs(h[−1]))+30))
cutoff_1_meas = np.argmin(np.abs(20*np.log10(np.abs(h)/np.abs(h[−1])) + 6))
k_6 = w[cutoff_1_meas]
k_30 = w[cutoff_0_meas]
c_max = f/k_30
c_min = f/k_6
r = k_6/k_30

In this case, the characteristic quantities of the filter are as follows: K₋₆-25.1 m⁻¹, K₋₃₀=5.6 m⁻¹ and r=4.48.

A filter can thus be generated according to the stiffness specification, but the length necessary to create the filter: 3.2 cm, will be lost for the visualization of shear wave velocities (shortening of the depth of the area of interest).

Of course, other Matlab or python functions (than the “fiwin2” function) can be used.

### 2.3.2. FIR Band-Pass Filter

In one embodiment of the invention, the filtering technique is based on a one-dimensional band-pass filter applied along the deformation.

Similar to the highpass filter defined in point 2.4.1, interest is given to the following quantities:
K₋₆ which corresponds to the value of the wavenumber (in number of cycles per meter) for which the attenuation of the filter is equal to 6 dB in the lower transition band,
the filter stiffness coefficient.

It should be noted that the corresponding stiffness coefficient on which a constraint similar to that of the highpass filter (that is to say stiffness coefficient less than or equal to 4.5) is imposed, is defined in this case on the lower transition band of the band-pass filter. No constraint is imposed on the stiffness coefficient in the upper transition band. The length of its impulse response should not exceed a few cm, with a typical size comprised between 1 cm and 3 cm, in order to limit boundary effects while maintaining a sufficiently large region in the medium of interest.

The reader will understand that many modifications can be made to the invention described above without materially departing from the new teachings and advantages described herein.

Therefore, all such modifications are intended to be incorporated within the scope of the appended claims.

1. An analysis method for analyzing a region of interest of a viscoelastic medium, the analysis method including a phase of acquiring signals representative of the movement of at least one shear-propagation component and of a static-deformation component, the acquisition phase comprising the following steps:
emitting acquisition signals in the viscoelastic medium successively over time, and
detecting and recording reception signals received from the viscoelastic medium, each reception signal being associated with a respective acquisition signal,
wherein the analysis method further comprises a processing phase for determining at least one property of the medium, said processing phase comprising the following steps:
determining images of the region of interest of the medium from the reception signals, each image being determined from a respective reception signal and comprising information representative of the region of interest at different times,
estimating images of movement by comparing images of the region of interest, each movement image including information representative of the shear-propagation component and a static-deformation component,
filtering the images of movement to attenuate the static-deformation component, and
determining said and at least one property of the medium from the filtered images of movement.

2. The analysis method according to claim 1, wherein the filtering step comprises the application of a finite impulse response filter the characteristics of which are defined according:
to an average frequency of the shear-propagation component comprised between 10 Hz and 1500 Hz, and
to a maximum group velocity of the shear-propagation component in the region of interest.

3. The analysis method according to claim 2, wherein the finite impulse response filter comprises a passband and a stopband:
the passband being defined at least by a lower bound K₋₆ said lower bound K₋₆ being equal to a ratio between the average frequency of the shear component and the maximum group velocity of the shear component, the attenuation in the passband being less than or equal to 6 dB, and
the stopband being defined at least by a stop bound K₋₃₀ proportional to the lower bound K₋₆, the attenuation in the stopband being greater than or equal to 30 dB, and the ratio between the lower bound K₋₆ and the stop bound K₋₃₀ defining a stiffness coefficient r of the finite impulse response filter, said stiffness coefficient being comprised between 1 and 4.5.

4. The analysis method according to claim 2, wherein the minimum and maximum velocities are determined based on predefined minimum and maximum hardnesses in the region of interest.

5. The analysis method according to claim 2, wherein the average frequency of the shear component is estimated from a signal representative of a time spectrum of the images of movement.

6. The analysis method according to claim 2, which further comprises an excitation phase including a step of generating the shear-propagation component by applying to the viscoelastic medium an excitation having the form of a low frequency impulse which has a central frequency f comprised between 10 Hz and 1500 Hz.

7. The analysis method according to claim 2, wherein the finite impulse response filter is of the highpass type in a useful frequency range comprised between 0 and 50 m⁻¹.

8. The analysis method according to claim 7, wherein said finite impulse response filter is obtained by combining an all-pass type filter and a lowpass type filter in said useful frequency range.

9. The analysis method according to claim 8, wherein the lowpass filter is a rectangular window lowpass filter or a filter whose impulse response is such that its maximum value is less than or equal to 2 times its average value.
